(19) United States
(12) Patent Application Publication
(10) Pub. No.: US 2005/0142166 A1
(43) Pub. Date: Jun. 30, 2005

(54) Peptides with anti-hypertensive properties

(75) Inventor: Benedicte Flambard, Frederiksberg (DK)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Chr. Hansen A/S

(21) Appl. No.: 10/956,084

(22) Filed: Oct. 4, 2004

Related U.S. Application Data
(63) Continuation-in-part of application No. PCT/DK03/00212, filed on Apr. 1, 2003.

(30) Foreign Application Priority Data
Apr. 3, 2002 (DK) PA 2002 00559
Aug. 9, 2002 (DK) PA 2002 01195

(51) Int. Cl. A61K 38/17; A23C 9/12; C12N 1/20; A61K 47/00

(52) U.S. Cl. 424/439; 514/2; 435/252.9; 426/34; 530/324

(57) ABSTRACT

A process for preparing peptides with anti-hypertensive properties and a process for preparing a functional food product comprising peptides with anti-hypertensive properties.

### CROSS-REFERENCE TO RELATED APPLICATIONS

This application is a continuation-in-part of PCT Application PCT/DK03/00212, filed Apr. 1, 2003, which claimed priority from Danish applications PA 2002 00559, filed Apr. 3, 2002 and PA 2002 01195, filed Aug. 9, 2002. The entire contents of the PCT and Danish applications are incorporated herein by reference.

### FIELD OF THE INVENTION

The present invention relates a process for preparing peptides with anti-hypertensive properties and a process for preparing a functional food product comprising peptides with anti-hypertensive properties.

### DESCRIPTION OF THE BACKGROUND ART

Hypertension (high blood pressure) has been reported to be one of the most important risk factors associated with heart attack in industrialized countries. Hypertension is frequently treated with drugs that strongly inhibit the angiotensin-converting enzyme (ACE). The prevention of high blood pressure in the early stage of the development of the disease, can be an alternative, to the treatment of hypertension with drugs. A large number of food-derived bioactive compounds are currently considered as beneficial for general well being or as health promoting (Aimutis, 2001 Bulletin of the IDF 363:30-38).

In the regulation of blood pressure, angiotensin I-converting enzyme (ACE) plays an important role. ACE acts to increase the blood pressure. In the renin-angiotensin system, ACE converts angiotensin-I to angiotensin-II by hydrolysing His-Leu from its C-terminal. Angiotensin II exhibits a strong vasoconstricting action. Additionally, in the kinin kallikrein system, ACE deactivates bradykinin, which aids vasodilation. ACE inhibitors are therefore useful in reducing blood pressure. Currently several ACE inhibitors already exist. The first reported ACE inhibitors were naturally occurring peptides found in snake venom (Ferreira et al. 1970 Biochemistry 9:2583-2592). Since then, many other ACE inhibitors hibitors have also been discovered (FitzGerald and Meisel, 2000 Br. J. Nutr. 58:33-37).

The article [Yamamoto et al (1994, J. Dairy Sci., 77: 917-922)] discloses that milk fermentation by Lactobacillus helveticus produced anti-hypertensive effects due to the liberation of peptides from casein in the milk by the proteolytic activity of L. helveticus. The peptides act as ACE inhibitors. The anti-hypertensive activity of these peptides was tested on spontaneously hypertensive rats. Milk fermentation by an isogenic mutant of Lb. helveticus that does not have proteolytic activity does not show any anti-hypertensive effects (Yamamoto, 1994 J. Dairy Sci. 77:917-922).

In the article [Nakamura Y. et al (1995. J. Dairy Sci., 78: 1253-1257]), it is suggested that the anti-hypertensive properties of fermented milk are mainly caused by the presence of the peptides Val-Pro-Pro and ile-Pro-Pro in the fermented milk. These peptides are generally resistant to degradation by digestive enzymes due to the presence of the Pro-Pro sequence at the C-terminal. A Lactobacillus helveticus is used for the fermentation of the milk.

The article [Gobbetti M. et al (2000, Appl Environ Microbiol, 66 (9), 3898-3904.], describes fermented milk containing ACE-inhibitory peptides that were produced by using either Lactococcus lactis subspecies cremoris FT4 or Lactobacillus delbrueckii subspecies bulgaricus SS1 to ferment the milk. The production of the peptides is attributed to the proteolytic teolytic activity of the bacteria.

### SUMMARY OF INVENTION

The problem to be solved by the present invention is to provide a process for preparing peptides with increased anti-hypertensive properties.

The solution is based on that the present inventors have identified that by fermenting a food material (preferably milk) with two different bacteria (a proteolytic and a lytic lactic acid bacterium), peptides with increased anti-hypertensive properties are produced. For further details, see table 2 of working example 4.

Accordingly, a first aspect of the invention relates to a process for preparing peptides with anti-hypertensive properties, the process comprising fermenting a food material, comprising animal milk proteins or vegetable proteins, with a proteolytic lactic acid bacterium and a lytic lactic acid bacterium to obtain a fermented food material which comprises the peptides with anti-hypertensive properties.

A bacterium is considered lytic when it is capable of performing an autolysis of itself. Autolysis means breaking down or dissolution of the bacterium cell.

A bacterium is considered proteolytic when it is capable of synthesizing an active cell wall proteinase. In other words, capable of providing a proteinase, which is active outside the intracellular part of the bacterium. Further, the proteinase should have a specificity making it capable of degrading proteins (e.g. casein comprised in milk) to obtain peptides with anti-hypertensive properties.

Without being limited to theory, it is believed that the present invention operates in the following manner. The main ACE inhibitors are characterized as small peptides, with a molecular weight less than 3000 Da. In contrast, the peptidic fragments resulting from e.g. casein hydrolysis by cell wall proteinase (of a proteolytic bacterium) are in general larger than 3000 Da. Peptidases, by their large number and specificities, can degrade almost all types of peptides. However, the intracellular location of these enzymes prevents their access to non-transported peptides. It is therefore considered that the introduction of a lytic microorganism liberates these peptidases, which can then further hydrolyze the larger peptides previously hydrolyzed by the proteolytic strain to produce peptides which are suitable ACE inhibitors (peptides with increased anti-hypertensive properties).

Peptides produced as described herein, may be used to prepare a functional food product with anti-hypertensive properties.

Accordingly, a second aspect the invention relates to a process for preparing a functional food product comprising peptides with anti-hypertensive properties, the process comprising following steps:
(i) preparing a fermented food material according to a process for preparing peptides with anti-hypertensive properties, as described herein, and
(ii) packing it in a suitable way to get a functional food product.

The term “packing” should be understood broadly. It denotes that once a food material is fermented and a fermented food material is obtained, the fermented food material should be packed so that it could be provided to the consumer. It may be packed in a bottle, a tetra-pack, etc. Preferably, on the package or in corresponding marketing material is indicated that the functional food product has anti-hypertensive properties.

The process of the second aspect, illustrates one of the advantages of the processes as described herein. The use of the proteolytic and lytic bacteria provides directly after the fermentation a useful amount of peptides with very good anti-hypertensive properties. Consequently, it is not considered necessary to further purify or up-concentrate the peptides from the fermented food material. The fermented food material may be packed directly and provided to the market as a functional food product.

In a third aspect the invention relates to peptides with anti-hypertensive properties obtainable by a process for preparing peptides with anti-hypertensive properties as described herein.

Peptides, produced by fermentation with both a proteolytic and a lytic bacterium, are different from peptides produced by fermentation with a proteolytic bacterium only. This may be verified functionally by the improved anti-hypertensive effects of the peptides produced by fermentation with both a proteolytic and a lytic bacterium. See e.g. table 2 of working example 4 herein for further details.

In a fourth aspect the invention relates to a functional food product comprising peptides with anti-hypertensive properties obtainable by a process for preparing a functional food product as described herein.

In a fifth aspect the invention relates to use of peptides with anti-hypertensive properties of the third aspect for the manufacture of a medicament for the treatment of hypertension.

In a sixth aspect the invention relates to use of a functional food product comprising peptides with anti-hypertensive properties of the fourth aspect for the manufacture of a medicament for the treatment of hypertension.

Embodiments of the present invention are described below, by way of examples only.

### DETAILED DESCRIPTION OF THE INVENTION

Food Material

The food material should comprise animal milk proteins or vegetable proteins.

Preferably it comprises animal milk proteins preferably enumerated by, for example milk protein components, such as whole or defatted animal milk or milk casein.

Food material with vegetable proteins may preferably be enumerated by, for example corn, corn protein, wheat, wheat protein, soybean, defatted soybean or soybean protein.

Lactic Acid Bacterium

The term “lactic acid bacteria” denotes herein a group of Gram-positive, non-sporing bacteria, which carry out a lactic acid fermentation of sugars.

Among others, it includes species of lactic acid bacteria belonging to genus Lactobacillus, such as Lactobacillus helveticus, Lactobacillus delbruekii subsp. bulgaricus, etc., lactic acid bacteria belonging to genus Lactococcus, such as Lactococcus lactis, lactic acid bacteria belonging longing to genus Streptococcus, such as Streptococcus salivarius subsp. thermophilus, lactic acid bacteria belonging to genus Leuconostoc, such as Leuconostoc lactis, lactic acid bacteria belonging to genus Bifidobacterium, such as Bifidobacterium longum or Bifidobacterium breve, and lactic acid bacteria belonging to genus Pediococcus.

The lactic acid bacteria may be used as a mixture with other microorganisms, e.g. yeasts.

Lytic Lactic Acid Bacterium

As described above a bacterium is considered lytic when it is capable of performing an autolysis of itself. Autolysis means breaking down or dissolution of the bacterium cell.

The lytic character of a bacterium may be determined by using the activity of the enzyme PepX. This enzyme is a peptidase which is located only inside the cell (it is not excreted or exported). Its activity may be measured by using a chromogenic substrate called Ala-Pro-pNA (Alanine-Proline-paranitroanilide). If the enzyme is active, the substrate is cleaved liberating the paranitroanilide compound. This compound has a yellow colour that can be read at 405 nm. Detecting an activity in the whey (or supernatant) which is by definition outside the cell, means that there has been lysis of the cell. Therefore, by measuring at 405 nm, the amount of paranitroanilide can be ascertained. From this, the amount of cell lysis can be calculated. As the amount of PepX expression from one lactic acid bacterium to another one does not change significantly, this test can be used to compare one bacterial strain to another in term of lysis properties.

A suitable protocol for determining the lytic character of a lactic acid bacterium may comprise following steps
(i) culturing overnight the bacterium in a suitable laboratory media at a suitable temperature to obtain a culture,
(ii) centrifuging of the culture,
(iii) taking 60 μg of the supernatant and incubate it with 40 μg of chromogenic substrate Alanine-Proline-paranitroanilide [L-Ala-Pro-pNa; initial concentration of 1.5 mM, pH 7.5] in a microtiter plate, at 37° C.,
(iv) measuring the kinetic of the liberation of the yellow colour paranitroanilide compound at OD405 nm for 3 hours, and
(v) estimating the lytic activity as the value of the slope of the kinetic assay expressed pressed by OD/min.

The term “a suitable media” in step (i) denotes a media, which is suitable for growth of the cell. Suitable for growth means that the culture should reach the stationary phase of growth after the overnight culture. The skilled person knows to identify a suitable media in relation to a specific lactic acid bacterium.

For a Lc. lactis bacterium a suitable media may be M17 and for Lb. helveticus a suitable media may be MRS. Reference is made to e.g. the standard textbook (Maniatis et al, “Molecular Cloning. A laboratory manual”. Cold Spring Harbor Laboratories, 2nd Edition/3 Volume, 1989), for further description of suitable medias.

A suitable temperature is a temperature that is suitable for growth of the bacterium. The skilled person knows how to identify this for a particular lactic acid bacterium. For Lactobaccilus species a suitable temperature is 37° C. and for Lactococcus species a suitable temperature is 30° C.

Preferably, the lytic lactic acid bacterium has a lytic activity within the range from 2.0 OD/min to 30 OD/min, more preferably within the range from 2.0 OD/min to 20 OD/min, and even more preferably within the range from 2.0 OD/min to 10 OD/min. Even more preferably the lytic lactic acid bacterium has a lytic activity within the range from 3.25 OD/min to 30 OD/min, more preferably within the range from 3.25 OD/min to 20 OD/min, and even more preferably within the range from 3.25 OD/min to 10 OD/min.

Further, the lactic acid bacterium is preferably mesophilic, i.e. with an optimal growing temperature of from 25° C. to 30° C.

Numerous different lactic acid bacteria are publicly available to the skilled person. Reference is e.g. made to the articles acknowledged in the background section above; EP583074, EP821968, EP1016709, WO01/32905, WO01/32836; Deutsche Sammlung von Mikroorganismen und Zellkulturen GmbH (DSMZ); and the Internet taxonomy browser of NCBI [at filing date available on http://www.ncbi.nlm.nih.gov/.].

In order to identify a suitable specific lytic bacterium, it is routine work for the skilled person to simply e.g. obtain 10 different publicly available bacteria and test whether they have lytic activity as described herein.

Preferably, the lytic lactic acid bacterium is a bacterium of the genus Lactococcus, more preferably it is a Lactococcus lactis strain.

A sample of a particular preferred Lactococcus lactis strain IG227 has been deposited at DSMZ (Deutsche Sammlung von Mikroorganismen und Zellkulturen GmbH) under the accession number DSM 14797 with a deposit date of 5 Feb. 2002. The deposit has been made under the conditions of the Budapest Treaty on the International Recognition of the Deposit of Microorganisms for the Purposes of Patent Procedure.

Accordingly, a particular preferred embodiment relates to a process as described herein where the lytic lactic acid bacterium is Lactococcus lactis with the registration number DSM 14797 or a mutant thereof.

Using as starting material the deposited DSM 14797 strain, the skilled reader can by conventional mutagenesis or re-isolation techniques obtain further mutants or derivatives which retain the lytic ability as described herein.

In this relation, a separate aspect of the invention relates to a Lactococcus lactis bacterium with the registration number DSM 14797 or a mutant thereof.

Proteolytic Lactic Acid Bacterium

As said above, a bacterium is considered proteolytic when it is capable of synthesizing an active cell wall proteinase. In other words, capable of proving a proteinase, which is active outside the intracellular part of the bacterium. Further, the proteinase should have a specificity making it capable of degrading proteins (e.g. casein comprised in milk) to obtain peptides with anti-hypertensive properties.

Preferably, the proteolytic activity of a bacterium is ascertained by a protocol comprising the steps:
(i) fermenting overnight 200 ml of a food material with the bacterium,
(ii) extracting the produced peptides, and
(iii) measuring the anti-hypertensive properties of the extracted peptides by an assay measuring the peptidic concentration required to inhibit 50% of the ACE activity.

The ACE inhibition activity assay is herein also termed DL50. The lower the DL50 value is the better is the anti-hypertensive effect of the peptides comprised in the fermented food material.

In step (i) of the protocol, the food material is preferably fresh milk. Further, the bacterium is preferably inoculated to the food material in the form of an overnight stock culture of (1% v/v) and maintained overnight at a suitable temperature. A suitable temperature is a temperature that is suitable for growth of the bacterium. The skilled person knows how to identify this for a particular lactic acid bacterium. For Lactobaccillus species a suitable temperature is 37° C. and for Lactococcus species a suitable temperature is 30° C.

In working example 2 herein is provided a detailed preferred protocol for the fermenting and extracting steps and in example 3 is a detailed preferred protocol for the DL50 ACE activity assay.

Preferably, the proteolytic lactic acid bacterium has a proteolytic activity making it capable of, in a protocol comprising the steps:
(i) fermenting overnight 200 ml of a food material with the bacterium,
(ii) extracting the produced peptides, and
(iii) measuring the anti-hypertensive properties of the extracted peptides by an assay measuring the peptidic concentration required to inhibit 50% of the ACE activity (DL50),

producing peptides with an angiotensin-converting enzyme (ACE) inhibition activity (DL50) of from 0.25 to 5.0 (mg/ml).

More preferably, the proteolytic lactic acid bacterium is capable of producing peptides with an angiotensin-converting enzyme (ACE) inhibition activity (DL50) of from 0.25 to 4.0 (mg/ml), and even more preferably the proteolytic lactic acid bacterium is capable of producing ducing peptides with an angiotensin-converting enzyme (ACE) inhibition activity (DL50) of from 0.25 to 3.5 (mg/ml).

The lower DL50 range may be, instead of 0.25 mg/ml, 1.0 mg/ml.

It is preferred that the proteolytic lactic acid bacterium does not have a too high lytic activity.

Accordingly, a preferred embodiment relates to a process, as described herein, wherein the proteolytic lactic acid bacterium has a lytic activity of less than 0.75 OD/min, preferably a lytic activity of less than 0.50 OD/min, and more preferably a lytic activity of less than 0.25 OD/min. The lytic activity is preferably measured as described above.

Further, the proteolytic bacterium is preferably thermophilic i.e. with an optimal growing temperature of from 38° C. to 55° C.

As described for the lytic bacterium, in order to identify a suitable specific proteolytic bacterium, it is routine work for the skilled person to simply e.g. obtain 10 different public available bacteria and test whether they have proteolytic activity as described herein. Further, the proteolytic bacterium could be tested for a preferred weak lytic activity.

Preferably, the proteolytic lactic acid bacterium is a bacterium of the genus Lactobacillus, in particular a Lactobacillus helveticus strain.

A suitable proteolytic Lactobacillus helveticus bacterium include a Lactobacillus helveticus LBK-16, DSM13137, bacterium (see WO01/32836); Lactobacillus helveticus bacterium JCM-1004 (see EP583074); Lactobacillus helveticus bacterium FERM BP-4835 (see EP821968); and Lactobacillus helveticus bacterium FERM BP-6060 (see EP1016709). A sample of a particular preferred Lactobacillus helveticus strain CHCC4080 has been deposited at DSMZ (Deutsche Sammlung von Mikroorganismen und Zellkulturen GmbH) under the accession number DSM 14997 with a deposit date of 15 May 2002. The deposit has been made under the conditions of the Budapest Treaty on the International Recognition of the Deposit of Microorganisms for the Purposes of Patent Procedure.

Accordingly, a particular preferred embodiment relates to a process as described herein where the proteolytic lactic acid bacterium is Lactobacillus helveticus with the registration number DSM 14997 or a mutant thereof.

Using as starting material the deposited DSM 14997 strain, the skilled reader can by conventional mutagenesis or re-isolation techniques obtain further mutants or derivatives which retain the proteolytic ability as described herein.

In this relation, a separate aspect of the invention relates to a Lactobacillus helveticus bacterium with the registration number DSM 14997 or a mutant thereof.

Further, a sample of another particular preferred Lactobacillus helveticus strain CHCC5951 has been deposited at DSMZ (Deutsche Sammlung von Mikroorganismen und Zellkulturen GmbH) under the accession number DSM 14998 with a deposit date of 15 May 2002. The deposit has been made under the conditions of the Budapest Treaty on the International Recognition of the Deposit of Microorganisms for the Purposes of Patent Procedure.

Fermentation

In the process of the present invention, the food material is fermented by lactic acid bacteria under operating conditions, which may be varied depending on the types of the food material and/or the combination of the lactic acid bacteria. Preferably, if the food material is not already an aqueous solution, food material is dissolved in a suitable aqueous solution, which is then admixed with lactic acid bacteria and cultivated by way of fermentation.

The culturing of the lactic acid bacteria may be performed by adding pre-cultured lactic acid bacteria starter to the food material medium, which may have been previously heat-sterilized and cooled to the predetermined temperature for incubation. The inoculation amount of the lactic acid bacteria starter may preferably be 10⁵ to 10⁷ cells of lactic acid bacteria/ml medium. The temperature for incubation is usually 20 to 50° C. and preferably 30 to 45° C. The incubation time is usually 3 to 48 hours and preferably 6 to 24 hours. Particularly, it is preferred to perform cultivation in the medium having pH in a range of 3.5 to 7, more preferably 5 to 6, in order to perform cultivation of lactic acid bacteria efficiently. Further, it is preferred to perform pH-stat cultivation maintaining pH in a range of 4 to 7. The incubation may be terminated, without restriction, when the number of lactic acid bacteria exceeds 10⁸ cells/ml.

In all aspects (also referred to herein as “embodiments”) of the invention, relative amounts of the proteolytic lactic acid bacterium to the lytic acid bacterium range from about 1:10 to about 10:1, by volume, including about 1:3 to about 3:1 by volume.

A preferred embodiment relates to a process, as described herein, wherein the fermenting of the food material is performed under conditions, which produce from 0.5 to 25 mg peptides with anti-hypertensive properties per 100 ml of the food material, more preferably which produce from 1 to 5 mg peptides with anti-hypertensive properties per 100 ml of the food material.

Subsequent Purification of the Anti-Hypertensive Peptides from the Fermented Food Material

As stated above, the use of the proteolytic and lytic bacteria, as described herein, provides directly after the fermentation a useful amount of peptides with very good anti-hypertensive properties.

However, in some circumstances it may be preferred to perform a subsequent purification of the anti-hypertensive peptides from the fermented food material. This may for instance be when the peptides are to be used in a pharmaceutical tablet, which requires a very high concentration of the anti-hypertensive peptides.

Accordingly, an embodiment of the invention relates to a process for preparing peptides with anti-hypertensive properties as described herein, wherein the fermented food material is further processed in a way that purifies or up-concentrates the peptides with anti-hypertensive properties.

For instance, the fermented food material containing peptides with anti-hypertensive properties may be centrifuged, and the resulting supernatant may be subjected to purifying treatment with a reverse-phase resin, for obtaining a sample in which the content of the peptides with anti-hypertensive properties is increased.

The centrifugation may preferably be performed, for example, at 2,000 to 20,000 rpm for 1 to 20 minutes. The centrifugation may also be performed in a centrifugator.

The purifying treatment with a reverse-phase resin may be performed by absorption and elution of the peptides with a reverse-phase resin, and/or by reverse-phase chromatography, thereby increasing purity of the peptides.

For further technical details in relation to this reverse-phase resin protocol reference is made to EP821968.

Alternatively, the fermented food material is further processed in a way wherein a nanofiltration is performed on the fermented food material. This may be done in order to remove lactic acid or monovalent ions from the fermented food material.

For further technical details in relation to this nanofiltration protocol reference is made to WO01/32905.

A Functional Food Product Comprising Pentides with Anti-Hypertensive Properties

As said above, it may be preferred to perform a subsequent purification of the anti-hypertensive peptides from the fermented food material.

Accordingly, an embodiment of the invention relates to a process for preparing a functional food product comprising peptides with anti-hypertensive properties, the process comprising following steps:
(i) preparing a fermented food material according to a process as described herein,
(ia) the fermented food material of step (i) is further processed in a way that purifies or up-concentrates the peptides with anti-hypertensive properties according to a process as described above,
(ib) the purified or up-concentrated peptides of step (ia) are then added to a food material, and
(ii) packing the resulting food material comprising the purified or up-concentrated peptides of step (ia) in a suitable way to get a functional food product.

Preferably, the food material of step (ib) is a fermented food material prepared according to a process as described herein. This corresponds to a situation where one wants a relatively high concentration of the peptides with anti-hypertensive properties in the functional food product.

Use and Preferred Doses of the Peptides with Anti-Hypertensive Properties

The peptides with anti-hypertensive properties, obtained by a process of the present invention tion, are usually a mixture of peptides, and may contain other peptides. For use as foods and drinks, the fermented food material containing the peptides and/or purified products thereof may be used directly. Alternatively, the fermented food material (also referred to herein as “agent”) may be powdered by freeze drying, spray drying or drum dryer drying, before use.

A preferred effective amount of the anti-hypertensive peptides of the present invention varies depending upon the age and condition of a person, and is in a range of 0.05 to 10 mg/kg body weight/day. It is preferable to administer 0.3 to 3.0 mg/kg body weight/day. If the dose is not less than 0.005 mg/kg body weight/day, sufficient effect may be expected. If the dose is not more than 10 mg/kg body weight/day, the effect may be exhibited efficiently.

The anti-hypertensive peptides may be used to treat hypertension by administering to a patient suffering from hypertension a medicament comprising the anti-hypertensive peptides. The medicament includes an effective amount of the anti-hypertensive peptides.

### EXAMPLES

If not otherwise mentioned, individual steps were performed using standard methods as e.g. described in the general textbooks (Maniatis, T., Fritsch, E. F., Sambrook, J. “Molecular Cloning. A laboratory manual”. Cold Spring Harbor Laboratories, 2nd Edition/3 Volume, 1989; Ausubel, F. M., et al. (eds.) “Current Protocols in Molecular Biology”. John Wiley and Sons, 1995.

### Example 1

### Identification of Suitable Proteolytic and Lytic Lactic Acid Bacteria

Different lactic acid bacterial strains were tested for their lytic activity and their proteolytic activity. The tests were performed as described above.

Lactobacillus species were streaked on MRS agar and incubated anaerobically for 48 h at 37° C. A single colony was picked, inoculated into MRS broth and grown overnight at 37° C. Lactococcus species were streaked on M17 agar and incubated aerobically for 48 h at 30° C. A single colony was picked, inoculated into M17 broth and grown overnight at 30° C. Stock cultures were prepared from these overnight cultures and were stored at −80° C. in 20% glycerol.

The strains used and their lytic, proteolytic properties are listed below in Table 1.

TABLE 1
Strains used and their lytic, proteolytic properties

| | | Proteolytic | |
|---|---|---|---|
| | | activity | Lytic |
| | Reference | (DL50) | activity |
| Species | number | (mg/ml) | (OD/min) |
| “Proteolytic strains” | | | |
| Lb. helveticus | CHCC637 | 3.32 | −0.253 |
| Lb. helveticus | CHCC4080 | 2.34 | −0.095 |
| Lb. acidophilus | CHCC2169 | 2.6 | −0.094 |
| Lc. lactis | CHCC3923 | 2.55 | −0.09 |
| “Lytic strains” | | | |
| Lc. lactis | CHCC3949 | >10 | 2.561 |
| Lc. lactis | CHCC3950 | >10 | 5.89 |
| Lc. lactis | CHCC3053 | >10 | 3.42 |

### Example 2

### Preparation of Fermented Milk and Extraction of Peptides

Fermentation is performed by inoculating 200 ml of fresh milk with an overnight stock culture of example 1 (1% v/v) and maintaining overnight at 37° C. or 30° C. dependent on the strain used. In the case of a mixed culture (a proteolytic and a lytic strain), the inoculum is 0.5%/0.5% of each strain (i.e. for a mixed culture the milk is also inoculated with 1% v/v of a bacterial culture)

From the fermented milk, extraction of the peptides may be achieved by using the following protocol:
Centrifuge at 3000 g for 10 min at room temperature.
Withdraw the supernatant and adjust to pH 8.3 (optimal pH for ACE activity test) with NaOH.
Centrifuge the obtained supernatant at 3000 g for 10 min at room temperature.
Withdraw the supernatant (whey), which comprises the peptides.
Determine the concentration of peptides in the whey by the Lowry test (mg peptide/ml whey) (Lowry et al, 1951. J. Biol. Chem., 193:265-275).

The whey may be used directly for ACE assay or freezed at −20° C. for later use, after it is suitably thawed. The whey comprising the peptides is termed “peptide solution” in example 3.

### Example 3

### ACE Activity Assay

The peptide pools of milk fermented are tested for ACE activity in vitro. The DL50 (mg/ml) is the peptidic concentration, which inhibits 50% of ACE activity. The lower this DL50 value is, the better the anti-hypertensive effect of the fermented milk. The ACE activity of the extracted peptides is measured by the following protocol:

The essence of the assay is that ACE degrades a hippuryl-L-histidyl-L-leucine (HHL) substrate and by adding a colour agent, a colour is developed in the assay. If peptides are present the peptides inhibit ACE and less HHL substrate is degraded. This means less colour is developed after addition of the colour agent.

Solution Preparation:

Incubation buffer: 188 mmol/l boric acid pH 8.3, 1.375 mmol/l potassium chloride.
(Dissolve 2.91 g of boric acid and 25.63 g potassium chloride in 200 ml of distilled water. Adjust the pH to 8.3 with 1 mol/l potassium hydroxide and dilute to 250 ml with distilled water. Store at room temperature).

Substrate solution: 5.8 mmol/l hippuryl-L-histidyl-L-leucine (HHL).
(Dissolve 250 mg hippuryl-L-histidyl-L-leucine in about 90 ml incubation buffer and fill up to 100 ml with the same buffer. Store at 40° C. The substrate solution can be used for at least 2 weeks).

Stop solution: 100 mmol/l HEPES pH 9, 2.5 mmol EDTA.
(Dissolve 23.83 g HEPES and 0.93 g EDTA in 800 ml distilled water. Adjust to pH 9 with 1 mol/l sodium hydroxide and dilute to 11 with distilled water. Store at room temerature.)

Colour reagent: 136 mmol/l cyanuric chloride in 1,4-dioxane.
(Dissolve 12.50 g cyanuric chloride in about 400 ml of 1,4-dioxane and fill up with 1,4-dioxane to 500 ml. Store at room temperature in dark-brown glass bottle).

Assay: (all solutions are equilibrated to room temperature)
Make a dilution series of the peptide solution with incubation buffer. The series consist of 6 dilutions going from the undiluted peptide solution to a blank (only incubation buffer)
For each of the dilutions, place 10 μl of peptide solution, 40 μl of substrate (HHL) solution (2.5 g/l) and 2.5 μl of ACE (0.25 Units/ml) in a glass tube.
The positive control comprises 2.5 μl ACE, 10 μl of incubation and 40 μl of substrate (HHL)
The negative control comprises 12 μl of incubation buffer and 40 μl of substrate (HHL)
Incubate at 37° C. for 1 hour.
Stop the reaction by adding 300 μl of stop solution, followed by 150 μl of colour reagent
Mix vigorously.
Allow to stand for 5 minutes and centrifuge at 3300 g for 30 min at room temperature to remove denatured protein and excess cyanuric chloride.
Transfer 300 μl of supernatant of each sample to microtiter plate hole.
Read at 405 nm against water as a blank.

The ACE inhibition percentage is expressed by the formula:

$$ACE inhibition activity = \frac{OD 405 nm positive control - OD 405 nm sample}{OD 405 nm positive control - OD 405 nm negative control}$$

Each dilution has its own ACE inhibition percentage value that gives a curve expressing the ACE inhibition percentage in function of the peptide concentration of the whey. DL50 (peptidic concentration that inhibits 50 % of ACE activity) is obtained by reading the peptidic concentration at the intersection point between the curve and the corresponding 50 % ACE inhibition point on the axis.

### Example 4

### ACE Activity of Peptides Obtained Using a Mixed Culture (a Proteolytic and Lytic Strain) During Fermentation

TABLE 2
Anti-hypertensive properties of fermented milk, where a mixed culture
(a proteolytic and lytic strain) is used during fermentation

| | Anti-hypertensive effect |
|---|---|
| | with DL 50 (mg/ml). Average |
| Culture used to ferment milk | of three determinations |
| Lb. helv.637 | 3.32 |
| Lb. helv.637/L. lactis 3949 | 2.76 |
| Lb. helv.637/L. lactis 3950 | 2.49 |
| Lb. helv.637/L. lactis IG227 | 1.84 |
| Lb. acido. 2169 | 2.6 |
| Lb. acido. 2169/L. lactis 3949 | 2.09 |
| Lb. acido. 2169/L. lactis 3950 | 1.82 |
| Lb. helv.4080 | 2.34 |
| Lb. helv.4080/L. lactis 3949 | 2.43 |
| Lb. helv.4080/L. lactis 3950 | 2.33 |
| Lb. helv.4080/L. lactis IG227 | 1.23 |
| Evolus ® (Valio, FI) product* | 1.7 |

*positive control - fermented milk which is commercially available as an antihypertensive product.

The addition of a lytic strain shows an enhanced anti-hypertensive effect of the fermented milk. For instance, the proteolytic Lb. helv. 4080 (alone) has effect 2.34 and L. lactis IG227 (alone) has no effect (DL 50 >10, see table 1). However, in combination the synergistic effect is 1.23.

1. A process for preparing peptides with anti-hypertensive properties, the process comprising fermenting a food material, comprising animal milk proteins or vegetable proteins, with a proteolytic lactic acid bacterium and a lytic lactic acid bacterium to obtain a fermented food material which comprises the peptides with anti-hypertensive properties.

2. The process of claim 1, wherein the food material comprises animal milk proteins.

3. The process of claim 2, wherein an animal milk protein is casein.

4. The process of claims 2 or 3, wherein the food material is milk or milk based material.

5. The process of any one of claims 1-3, wherein the lytic lactic acid bacterium has a lytic activity within the range from 2.0 OD/min to 30 OD/min, measured by an assay comprising:
(i) culturing overnight the bacterium in a suitable laboratory medium or media to obtain a culture,
(ii) centrifuging the culture,
(iii) taking 60 μl of a supernatant and incubating the supernatant with 40 μl of chromogenic substrate Alanine-Proline-paranitroanilide (L-Ala-Pro-pNa) in a microtiter plate, at 37° C.,
(iv) measuring the kinetic of the liberation of the yellow colour paranitroanilide compound at OD405 nm for 3 hours, and
(v) estimating the lytic activity as the value of the slope of the kinetic assay expressed pressed by OD/min.

6. The process of claim 5, wherein the initial concentration of the L-Ala-Pro-pNa is 1.5 mM and the initial pH is 7.5.

7. The process of any one of claims 1-3, wherein the proteolytic lactic acid bacterium has a lytic activity of less than 0.75 OD/min, measured by the assay of claim 5.

8. The process of any one of claims 1-3, wherein the proteolytic lactic acid bacterium has a proteolytic activity making it capable of producing peptides with an angiotensin-converting enzyme (ACE) inhibition activity (DL50) of from 0.25 to 5.0 (mg/ml), the peptides being produced by a process comprising:
(i) fermenting overnight 200 ml of a food material with the proteolytic lactic acid bacterium to produce a product comprising peptides;
(ii) extracting the peptides from the product; and
(iii) measuring the anti-hypertensive properties of the extracted peptides by an assay measuring the peptidic concentration required to inhibit 50% of the ACE activity (DL50).

9. The process of any one of claims 1-3, wherein the lytic lactic acid bacterium is a bacterium of the genus Lactococcus.

10. The process of claim 9, wherein the bacterium is a Lactococcus lactis strain.

11. The process of claim 10, wherein the strain is Lactococcus lactis strain IG227.

12. The process of any one of claims 1-3, wherein the proteolytic lactic acid bacterium is a bacterium of the genus Lactobacillus.

13. The process of claim 12, wherein the bacterium is a Lactobacillus helveticus strain.

14. The process of any one of claims 1-3, wherein the fermenting of the food material is performed formed under conditions which produce from 0.5 to 25 mg peptides with anti-hypertensive properties per 100 ml of the food material.

15. The process of any one of claims 1-3, wherein the fermenting of the food material is performed at 20 to 50° C. for 3 to 48 hours.

16. The process of any one of claims 1-3, wherein the fermenting of the food material is performed under conditions wherein the pH is in a range from pH 3.5 to 7.

17. The process of any one of claims 1-3, wherein the fermented food material is further processed to purify or up-concentrate the peptides with anti-hypertensive properties.

18. The process of claim 17, wherein the fermented food material is further processed by centrifuging and the obtained supernatant, comprising the peptides with anti-hypertensive properties, is isolated.

19. The process of claim 18, wherein the peptides are purified from the supernatant with a reverse-phage resin.

20. The process of claim 17, wherein the fermented food material is further processed by performing nanofiltration on the fermented food material.

21. A process for preparing a functional food product comprising peptides with anti-hypertensive properties, comprising:
(iii) preparing a fermented food material according to a process of any one of claims 1-3,and
(iv) packing the fermented food material to obtain a functional food product.

22. The process for preparing a functional food product of claim 21, additionally comprising between (i) and (ii):
(ia) further processing the fermented food material of step (i) to purify or up-concentrate the peptides with anti-hypertensive properties according to a process of claim 17, and
(ib) adding the purified or up-concentrated peptides of step (ia) to a food material.

23. The process of claim 22, wherein the food material of (ib) is the fermented food material prepared according to a process of any one of claims 1 to 3.

24. Peptides with anti-hypertensive properties obtained by a process of claims 17.

25. A functional food product comprising peptides with anti-hypertensive properties obtained by a process of claim 21.

26. A method of preparing a medicament for the treatment of hypertension, comprising combining the peptides of claim 24 with a pharmaceutically-acceptable carrier.

27. A method of preparing a medicament for the treatment of hypertension, comprising combining the functional food product of claim 25 with a pharmaceutically-acceptable carrier.

28. A Lactococcus lactis bacterium with the registration number DSM 14797 or a mutant thereof.

29. A Lactobacillus helveticus bacterium with the registration number DSM 14997 or a mutant tant thereof.

30. A method of treating hypertension comprising administering to a patient suffering from hypertension a medicament prepared by the process of claim 24.

31. A method of treating hypertension comprising administering to a patient suffering from hypertension the medicament prepared by the method of claim 27.

32. A method of treating hypertension comprising administering to a patient suffering from hypertension a medicament including the peptides prepared by the process of claim 1.

33. Peptides with anti-hypertensive properties prepared by a process of claim 1.
